(19) United States
(12) Patent Application Publication
(10) Pub. No.: US 2022/0372233 A1
(43) Pub. Date: Nov. 24, 2022

(54) TEMPLATE PARTICLES WITH MICROPORES AND NANOPORES

(71) Applicant: Fluent Biosciences Inc., Watertown, MA (US)

(72) Inventors: Yi Xue, Shrewsbury, MA (US); Jacob Ishibashi, Watertown, MA (US)

(21) Appl. No.: 17/747,569

(22) Filed: May 18, 2022

Related U.S. Application Data
(60) Provisional application No. 63/190,148, filed on May 18, 2021.

(51) Int. Cl. C08J 3/075; C12N 15/10; C08J 3/24; C08L 33/26; C08J 9/28

(52) U.S. Cl. CPC C08J 3/075; C12N 15/1017; C08J 3/24; C08L 33/26; C08J 9/28; C08J 2333/26

(57) ABSTRACT

The present invention includes compositions and methods for using and manufacturing hydrogel template particles with micropores and/or a nanoporous structure.

### BACKGROUND OF THE INVENTION

Biological fluids contain a variety of targets for diagnostic, research, and therapeutic purposes. These targets may include liquid biopsy targets, such as circulating cells (tumor, fetal, or stem), cellular components (e.g. nuclei), cell-free nucleic acids, extracellular vesicles, and antigens. Relevant targets also include infectious agents, such as prokaryotes, fungi, and viruses. However, the quantitative detection of biological targets, e.g., nucleic acids and proteins, at the single-cell and/or single-molecule level can be challenging due to the need to isolate and assay minute components in a sample.

Recently, an efficient and flexible target-specific approach to capture and label targets of interest from biological samples was described. The approach uses a particle-templated emulsification technique to capture and isolate biomolecules from a sample. Hatori et al., 2018 “Particle-Templated Emulsification for Microfluidics-Free Digital Biology” Anal. Chem., 10.1021/acs.analchem.8b01759. In short, the technique, also known as pre-templated instant partitions (PIPs) encapsulation, uses template particles to capture targets of interest in a sample. The template particles with captured targets are vortexed in immiscible fluids to create monodispersed droplets that contain a single template particle with the attached target. In certain variations of the technique, assays are performed on or using the captured targets in the monodispersed droplets. Such techniques can be improved by providing needed reagents to the monodispersed droplets via the template particles. This, of course, necessitates that the reagents be accessible for their use. Accordingly, the present invention provides methods for improved access to relevant target molecules.

### SUMMARY OF THE INVENTION

The present disclosure relates to improved template particles for use in pre-templated instant partition (PIP) encapsulation, which can be used to create monodispersed droplets that contain a single template particle, a target of interest, and one or more necessary reagents for carrying out a desired biological assay. The present Inventors discovered that certain factors can be manipulated during the manufacture of the template particles to alter their internal structures such they include micro- and/or nanoporous structures. These structures define internal volumes within the templated particles. When the templated particles are loaded with reagents for a bioassay, these internal volumes allow access to the loaded reagents.

In PIP encapsulation, the template particles serve as templates for making a large number of monodisperse emulsion droplets simultaneously in a single tube or vessel. By adding a plurality of template particles into an aqueous mixture, layering oil over the aqueous phase, and vortexing or shaking the tube, the particles serve as templates while the shear force of the vortexing or shaking causes the formation of water-in-oil monodisperse droplets with one particle in each droplet.

PIP encapsulation relies on the template particle to define an accessible volume in the water-in-oil emulsion. In template particles without interior volumes, this accessible volume is defined by the aqueous phase between the template particle surface and the oil-water interface. This volume can be miniscule when compared to the total volume of the template particles. Moreover, only those loaded reagents close to the surface of the particles can be accessed during a bioassay. The present Inventors discovered that this limited accessible volume and access to loaded reagents can sometimes compromise the efficiency of capturing target molecules and performing bioassays using template particles.

The presently-disclosed template particles with micro- and/or nanoporous structures improve PIP encapsulation-based assays by significantly enlarging the accessible volume in the emulsion and allowing unparalleled access to reagents loaded in the particles.

The present invention provides a composition comprising a plurality of hydrogel particles suspended in an aqueous liquid. Each hydrogel particle comprises a mesh of cross-linked polymers and includes: (i) a plurality of micropores extending through the mesh of cross-linked polymers, each micropore having an open interior volume having a dimension of at least about a micron; and/or a nanoporous structure in the mesh of cross-linked polymers, wherein the hydrogel mesh has a mesh size of at least about 200 nm, wherein the nanoporous structure comprises at least one open interior volume in the hydrogel mesh. In certain aspects, the particles are loaded with a reaction reagent. The aqueous liquid can permeate the interior volume of the micropores and/or nanoporous structure allowing analytes in the fluid to access the reagent and/or for the loaded reagents to flow out of the template particle and react with analytes in the fluidic droplets.

In certain aspects, each particle includes a plurality of micropores. Each particle may further include a nanoporous structure. In certain aspects, each particle includes a nanoporous structure.

Particles of the invention can be loaded with reagents. Suitable reagents include, for example, one or more of enzymes, enzyme cofactors, nucleotides, polynucleotides, amino acids, peptides, proteins, probes, primers, salts, ions, buffers, labels, dyes, antibodies, polymers, and carbohydrates. In certain aspects, the reagents include one or more target capture moiety. The target capture moiety, for example, captures one or more of circulating cells, cellular components, cell-free nucleic acids, extracellular vesicles, protein antigens, prokaryotic cells, fungi, viruses, and combinations thereof. In certain aspects, the reagents include reagents for one or more of nucleic acid synthesis, transcription, reverse transcription, and cell lysis. One or more of the reagents can be covalently linked to a particle.

The present invention also provides methods for performing bioassay s using the templated particles. In certain aspects, a method includes combining template particles with samples in a first fluid, wherein the template particles are hydrogel particles suspended in the first fluid, each hydrogel particle comprising a mesh of cross-linked polymers. Each hydrogel particle is loaded with a reaction reagent and includes: (i) a plurality of micropores extending through the mesh of cross-linked polymers, each micropore having an open interior volume having a dimension of at least about a micron; and/or (ii) a nanoporous structure in the mesh of cross-linked polymers, wherein the hydrogel mesh has a mesh size of at least 200 nm in length, wherein the nanoporous structure comprises at least one open interior volume in the hydrogel mesh. In certain aspects, the method further includes adding a second fluid immiscible to the first fluid and shearing the fluids to generate a plurality of monodispersed droplets simultaneously that contain a single one of the template particles and one or more of the samples.

In certain aspects, the first liquid permeates the interior volume of the micropores and/or nanoporous structure allowing analytes in the first fluid to access the reaction reagent. The interior volume is, for example, occupied by at least the first fluid and one or more of the reagents.

In certain aspects, the samples include at least one of circulating cells, cellular components, cell-free nucleic acids, extracellular vesicles, protein antigens, prokaryotic cells, fungi, viruses, and combinations thereof. The samples are cells and the sample in each of the plurality of monodisperse droplets is a single cell.

The present invention also provides methods for producing cross-linked template particles. An exemplary method includes, for example, preparing an aqueous phase fluid comprising an acrylamide/bisacrylamide copolymer matrix, wherein the aqueous phase fluid comprises at least about 3.5 wt % acrylamide/bisacrylamide; and co-flowing the aqueous phase fluid and a fluid immiscible to the aqueous phase fluid through a droplet generation device. In certain aspects, the resulting template particles have an effective hydrogel mesh size less than 200 nm in length.

In certain aspects, the effective hydrogel mesh size is modulated via a ratio of acrylamide monomers to bisacrylamide monomers in the copolymer matrix.

The present disclosure also provides methods for producing cross-linked template particles that includes preparing an aqueous phase fluid comprising acrylamide monomers and PEG; and co-flowing the aqueous phase fluid and a fluid immiscible to the aqueous phase fluid through a droplet generation device. In certain aspects, the resulting template having an open interior volume having a dimension of at least about a micron. The aqueous phase fluid includes at least about 4 wt % to about 2 wt % PEG. In certain aspects, the method further includes washing the PEG from the template particles.

### BRIEF DESCRIPTION OF THE DRAWINGS

FIG. 1 shows a schematic showing a cross-section of a template particle isolated in a monodisperse oil-in-water droplet.

FIG. 2 shows a schematic showing a cross-section of an exemplary template particle isolated in a monodisperse oil-in-water droplet.

FIG. 3 shows a schematic showing a cross-section of an exemplary template particle isolated in a monodisperse oil-in-water droplet.

FIG. 4 shows a schematic showing a cross-section of an exemplary template particle isolated in a monodisperse oil-in-water droplet.

FIG. 5 shows a schematic showing a cross-section of an exemplary capture template particle.

FIG. 6 shows the template particle from FIG. 5 isolated in a monodisperse droplet with the captured target.

FIG. 7 provides a microscope image of exemplary 3.5% PAA template particles with a nanoporous structure in the hydrogel.

FIG. 8 provides a microscope image of an exemplary template particle with a microporous structure made using 4% PAA and 2% PEG20K.

FIG. 9 provides a microscope image of an exemplary template particle with a microporous structure made using 6% PAA and 4% PEG20K.

FIG. 10 shows the results of manufacturing template particles with a nanoporous structure.

FIG. 11 shows the results of manufacturing template particles with a nanoporous structure.

FIG. 12 shows the results an amplification and sequencing assay using various types of template particle.

FIG. 13 provides a saturation curve comparing the median genes read per cell versus targeted sequencing depth (reads/cell) for different types of template particles.

FIG. 14 provides a saturation curve comparing the median UMIs read per cell versus targeted sequencing depth (reads/cell) for different types of template particles.

### DETAILED DESCRIPTION OF THE INVENTION

The present disclosure relates to improved templated particles for use in pre-templated instant partition (PIP) encapsulation, which can be used to create monodispersed droplets that contain a single template particle, a target of interest, and one or more necessary reagents for carrying out a desired biological assay. The templated particles are manufactured such that they include micro- and/or nanoporous structures. These structures define internal volumes within the template particles. When the templated particles are loaded with reagents for a bioassay, these internal volumes allow access to the loaded reagents.

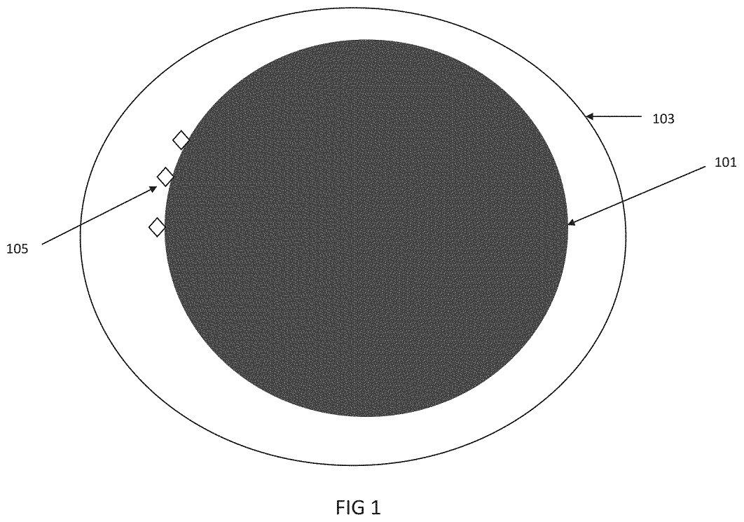

FIG. 1 shows a schematic showing a cross-section of a templated particle 101 isolated in a monodisperse oil-in-water droplet 103. This template particle lacks either nanopores or micropores, and thus no accessible internal volume. Since the templated particle 101 does not contain an interior volume, the accessible volume in the oil-in-water droplet is between the surface of the template particle 101 and the oil-water interface of the droplet 103. Consequently, if the templated particle is loaded with reagents for a particular bioassay, only those reagents 105 located near the surface of the template particle 101 are available for the assay.

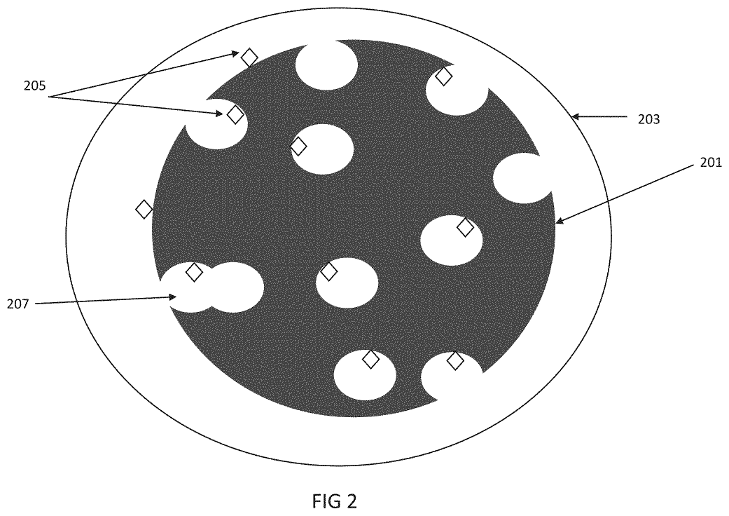

FIG. 2 shows a schematic showing a cross-section of an exemplary templated particle 201 isolated in a monodisperse oil-in-water droplet 203. The templated particle 201 is made, either wholly or in part, from a cross-linked hydrogel and includes a plurality of micropores 207 in the hydrogel of the particle. These micropores are micron-scale voids in the template particle hydrogel. In certain aspects, these micropores may connect to one another and permeate the interior of the template particle. The particle also includes one or more loaded reagents 205. The reagents 205 may be disposed on the outer surface of the template particle 201 and/or in one or more of the micropores 207. The micropores 207 provide an accessible interior volume in the template particle 201. Consequently, fluid and/or analytes in the monodisperse droplet 203 can access the reagents 205 loaded within the interior of the template particle. Alternatively or additionally, fluid and/or analytes can access the reagents 205 in the interior prior to formation of the monodisperse droplet.

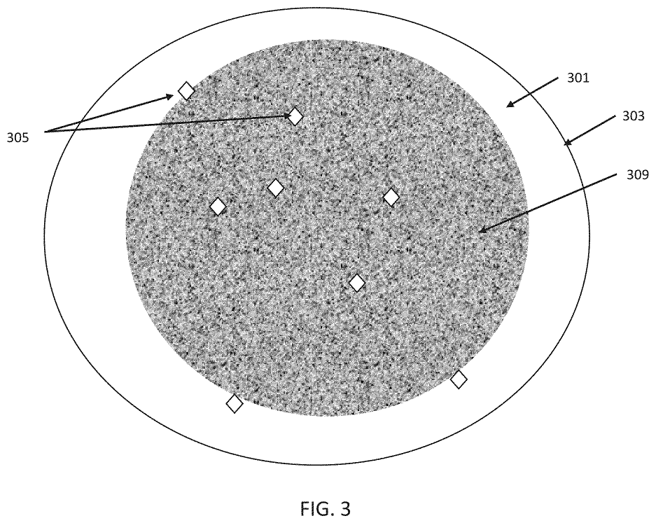

FIG. 3 shows a schematic showing a cross-section of an exemplary template particle 301 isolated in a monodisperse oil-in-water droplet 303. The template particle 301 is made, either wholly or in part, from a cross-linked hydrogel and includes a nanoporous structure 309 in the hydrogel of the particle. The cross-linked hydrogel forms a mesh of cross-linked polymers and has a mesh size of at least 200 nm in length and defines the nanoporous structure 309. In certain aspects, the nanoporous structure 309 provides interconnected voids that permeate the interior of the template particle 301. The particle also includes one or more loaded reagents 305. The reagents 305 may be disposed on the outer surface of the template particle 301 and/or within the nanoporous structure 309. The nanoporous structure 309 defines an accessible interior volume in the template particle 301. Consequently, fluid and/or analytes in the monodisperse droplet 303 can access the reagents 305 loaded within the interior of the template particle. Alternatively or additionally, fluid and/or analytes can access the reagents 305 in the interior prior to formation of the monodisperse droplet.

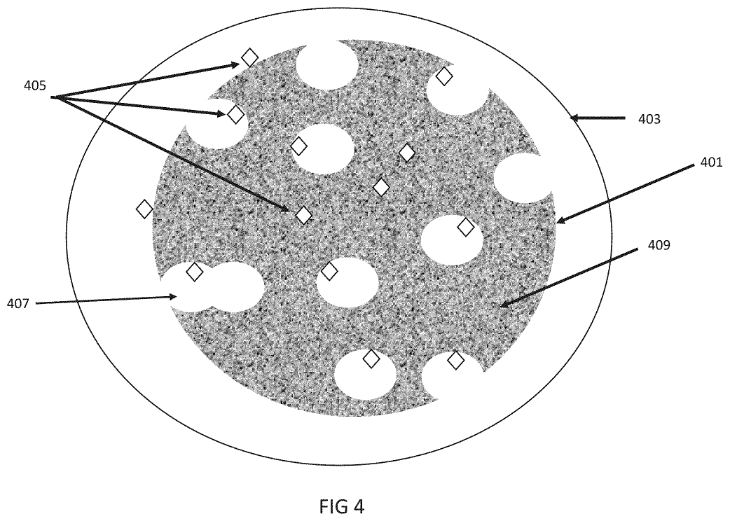

FIG. 4 shows a schematic showing a cross-section of an exemplary template particle 401 isolated in a monodisperse oil-in-water droplet 403. The template particle 401 is made, either wholly or in part, from a cross-linked hydrogel and includes a nanoporous structure 409 in which micropores 407 are disposed. The particle also includes one or more loaded reagents 405. The reagents 405 may be disposed on the outer surface of the template particle 401, within the nanoporous structure 409, and/or within the micropores 407. Consequently, fluid and/or analytes in the can access the reagents 405 loaded within the interior of the template particle via the nanoporous structure and/or the micropores.

In certain aspects, the template particles are capture template particles. Capture template particles include one or more reagents that capture targeted component from a sample.

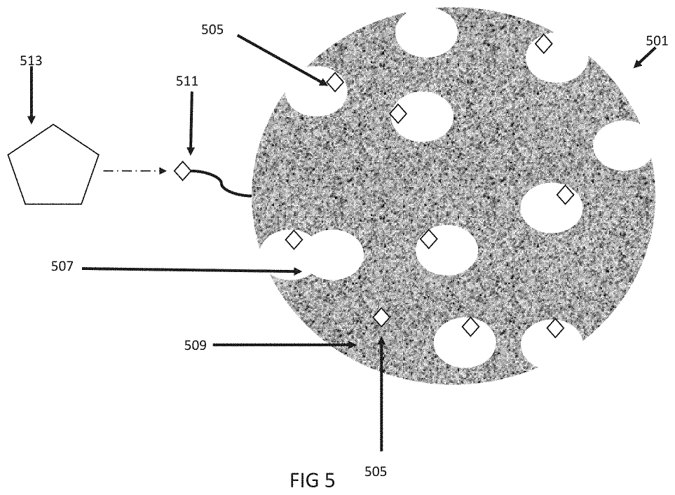

FIG. 5 shows a schematic showing a cross-section of an exemplary capture template particle 501. The capture template particle includes a nanoporous structure 509 in which micropores 507 are disposed. However, template particles with either only micropores or a nanoporous structure can be used. The template particle includes a capture element 511, which may be tethered to the template particle 501. The capture element can capture a target 513 in a sample. The target 513 may include, for example, a cell (e.g., circulating cells and/or circulating tumor cells), viruses, polynucleotides (e.g., DNA and/or RNA), polypeptides (e.g., peptides and/or proteins), and many other components that may be present in a biological sample. In certain aspects, the template particle has multiple, different capture elements 511 to capture multiple targets in a sample.

In certain aspects, the capture template particles are combined with target particles (e.g., biological sample components). The mixture of capture template particle and target particles is incubated for a sufficient amount of time to allow target-specific association of the target particles with the capture elements. Agitation or mixing can be used to increase the probability of target-specific association. To form encapsulations (also referred to herein as “partitions”), the mixture comprising the bound target particles and capture template particles is combined with a second fluid to provide a new mixture, wherein the second fluid is immiscible with the mixture comprising the bound target particles and capture template particles. In some embodiments, the second fluid is an oil. The next step includes shearing the new mixture such that a plurality of monodisperse droplets is formed. In certain aspects, a portion of the monodisperse droplets comprise a capture template particle. In some aspects, each capture template particle, whether or not associated with a target particle, is consequently encapsulated in monodisperse droplets.

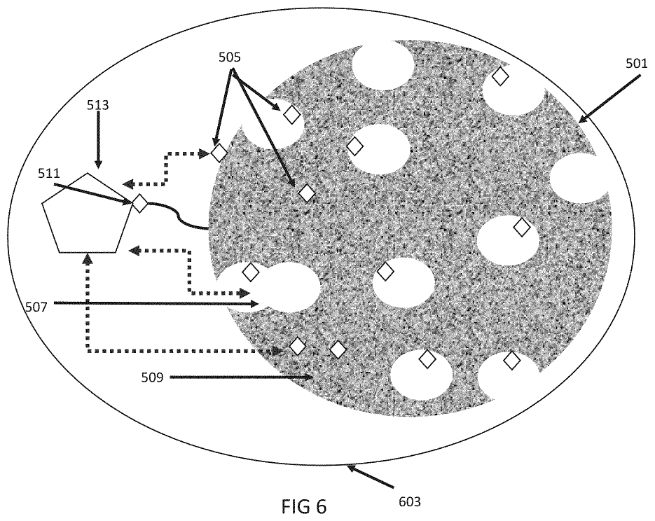

FIG. 6 shows the template particle 501 isolated in a monodisperse droplet 603 with the captured target 513. Fluid, analytes, and/or the target can permeate the nanoporous structure 509 and/or the micropores 507 to interact with the reagents 505 loaded in the template particle 501. Alternatively or additionally, the loaded reagents 505 can be released from the template particle via the nanoporous structure 509 and/or the micropores 507 to interact with the target 513. In certain aspects, the target is treated so as to release one or more components that react with the loaded reagents. For example, the target 513 may be a cell which is lysed in the droplet 603 to release components, e.g., nucleic acids, that react with the loaded reagents 505 to accomplish a particular assay.

In certain aspects, lysis may be induced by a stimulus, such as, for example, lytic reagents, detergents, or enzymes that are loaded into the template particles and released via the micropores and/or nanoporous structure. Lysing can additionally or alternatively involve heating the monodisperse droplets to a temperature sufficient to release lytic reagents contained inside the template particles into the monodisperse droplets.

In certain aspects, the capture moiety is a capture probe used to capture one or more nucleic acids from a sample. Nucleic acids that bind to capture probes may be subsequently amplified and/or reverse transcribed to form cDNA. An exemplary capture probe may include a linker region to allow covalent bond with the template particle, a primer region, which may be a universal primer region, a primer nucleotide sequence, one or more barcode regions, which may include an index sequence, and/or a unique molecular identifier (UMI), a random capture sequence, a poly-T capture sequence, and/or a target-specific capture sequence.

The term barcode region may comprise any number of barcodes, index or index sequence, UMIs, which are unique, i.e., distinguishable from other barcode, or index, UMI sequences. The sequences may be of any suitable length which is sufficient to distinguish the barcode, or index, sequence from other barcode sequences. A barcode, or index, sequence may have a length of 4, 5, 6, 7, 8, 9, 10, 11, 12, 13, 14, 15, 16, 17, 18, 19, 20, 21, 22, 23, 24, 25 nucleotides, or more. In some embodiments, the barcodes, or indices, are pre-defined and selected at random.

UMIs are advantageous in that they can be used to correct for errors created during amplification, such as amplification bias or incorrect base pairing during amplification. For example, when using UMIs, because every nucleic acid molecule in a sample together with its UMI or UMIs is unique or nearly unique, after amplification and sequencing, molecules with identical sequences may be considered to refer to the same starting nucleic acid molecule, thereby reducing amplification bias. Methods for error correction using UMIs are described in Karlsson et al., 2016, Counting Molecules in cell-free DNA and single cells RNA”, Karolinska Institutet, Stockholm Sweden, incorporated herein by reference.

To generate the capture template particles, target-specific elements are attached to the sized template particles. The target-specific elements of the present disclosure are selected from target-specific capture elements, and target-specific capture element genetic identifier. The target-specific capture elements can comprise, for example, Poly-T polynucleotide sequences, aptamers, and antibodies. In some embodiments, the target-specific capture elements comprise streptavidin, and may therefore attach to the capture moiety by biotin-streptavidin affinity.

The terms “nucleic acid amplification reagents” or “reverse transcription reagents” encompass without limitation one or more of dNTPs (mix of the nucleotides dATP, dCTP, dGTP and dTTP), buffer/s, detergent/s, or solvent/s, as required, and suitable enzyme such as polymerase or reverse transcriptase. The polymerase used in the presently disclosed targeted library preparation method may be a DNA polymerase, and may be selected from, but is not limited to, Taq DNA polymerase, Phusion polymerase, or Q5 polymerase. The reverse transcriptase used in the presently disclosed targeted library preparation method may be for example, Moloney murine leukemia virus (MMLV) reverse transcriptase, or maxima reverse transcriptase.

The present disclosure provides an improved emulsion droplet-based target capture and barcoding method. The present disclosure further provides capture template particles which allow capturing targets of interest from biological samples, and barcoding of specific nucleic acids contained in the captured targets. The nucleic acids can be contained within living or nonliving structures, including particles, viruses, and cells. The nucleic acids can include, e.g., DNA or RNA, which can then be detected, quantitated and/or sorted, e.g., based on their sequence as detected with nucleic acid amplification techniques, e.g., PCR and/or MDA. The disclosed methods involve the use of the capture template particles to template the formation of monodisperse droplets.

As used herein, the term “biological sample” or “sample” encompasses a variety of sample types obtained from a variety of sources, generally the sample types contain biological material. For example, the term includes biological samples obtained from a mammalian subject, e.g., a human subject, and biological samples obtained from a food, water, or other environmental source, etc. The definition encompasses blood and other liquid samples of biological origin, as well as solid tissue samples such as a biopsy specimen or tissue cultures or cells derived therefrom and the progeny thereof. The definition also includes samples that have been manipulated in any way after their procurement, such as by treatment with reagents, solubilization, or enrichment for certain components, such as polynucleotides. The term “biological sample” encompasses a clinical sample, and also includes cells in culture, cell supernatants, cell lysates, cells, serum, plasma, biological fluid, and tissue samples. “Biological sample” includes cells, e.g., bacterial cells or eukaryotic cells; biological fluids such as blood, cerebrospinal fluid, semen, saliva, and the like; bile; bone marrow; skin (e.g., skin biopsy); and antibodies obtained from an individual. Some non-limiting examples of a biological sample include liquid biopsy targets such as circulating cells (tumor, fetal, or stem), cellular components (e.g. nuclei), cell-free nucleic acids, extracellular vesicles, and protein antigens which are being targeted for development of non-invasive diagnostics for a variety of cancers. The term biological sample also includes biological targets indicative of disease such as prokaryotes, fungi, and viruses.

As described more fully herein, in various aspects the subject methods may be used to detect a variety of components from such biological samples. Components of interest include, but are not necessarily limited to, cells (e.g., circulating cells and/or circulating tumor cells), viruses, polynucleotides (e.g., DNA and/or RNA), polypeptides (e.g., peptides and/or proteins), and many other components that may be present in a biological sample.

A feature of certain methods as described herein is the use of a polymerase chain reaction (PCR)-based assay to detect the presence of certain oligonucleotides and/or genes, e.g., oncogene(s) present in cells. Examples of PCR-based assays of interest include, but are not limited to, quantitative PCR (qPCR), quantitative fluorescent PCR (QF-PCR), multiplex fluorescent PCR (MF-PCR), digital droplet PCR (ddPCR) single cell PCR, PCR-RFLP/real time-PCR-RFLP, hot start PCR, nested PCR, in situ polony PCR, in situ rolling circle amplification (RCA), bridge PCR, picotiter PCR, emulsion PCR and reverse transcriptase PCR (RT-PCR). Other suitable amplification methods include the ligase chain reaction (LCR), transcription amplification, self-sustained sequence replication, selective amplification of target polynucleotide sequences, consensus sequence primed polymerase chain reaction (CP-PCR), arbitrarily primed polymerase chain reaction (AP-PCR), degenerate oligonucleotide-primed PCR (DOP-PCR) and nucleic acid based sequence amplification (NABSA).

A PCR-based assay may be used to detect the presence of certain gene(s), such as certain oncogene(s). In such assays, one or more primers specific to each gene of interest are reacted with the genome of each cell. These primers have sequences specific to the particular gene, so that they will only hybridize and initiate PCR when they are complementary to the genome of the cell. If the gene of interest is present and the primer is a match, many copies of the gene are created. To determine whether a particular gene is present, the PCR products may be detected through an assay probing the liquid of the monodisperse droplet, such as by staining the solution with an intercalating dye, like SybrGreen or ethidium bromide, hybridizing the PCR products to a solid substrate, such as a bead (e.g., magnetic or fluorescent beads, such as Luminex beads), or detecting them through an intermolecular reaction, such as FRET. These dyes, beads, and the like are each example of a “detection component,” a term that is used broadly and generically herein to refer to any component that is used to detect the presence or absence of nucleic acid amplification products, e.g., PCR products.

The present invention also provides methods for manufacturing template particles with micropores and/or nanoporous structures.

The present Inventors have made the surprising discovery that both the microporosity and nanoporosity of template particles can be controlled by modifying one or more factors during template particle manufacture. In this context, nanoporosity refers to the effective cross-linking density of cross-linked polymers in the hydrogel of the particles, such that the hydrogel has an effective mesh size less than 200 nm in length. Microporosity refers to the micron scale structural features (i.e., micropores) in the hydrogel of the particle.

In an exemplary method, template particles with nanoporous structures can be manufactured by preparing a hydrogel in an aqueous phase fluid. The aqueous phase fluid with the hydrogel is co-flowed with a fluid immiscible to the aqueous phase, such as an oil, through a droplet generation device.

The Inventors discovered that the degree of nanoporosity in the hydrogel of a template particle can be controlled by modulating one or more factors, including total polymer loading in each template, the ratio of cross-linking agents in the hydrogel, and the chemical structures and interactions of monomers and crosslinkers in the hydrogel. The inventors have discovered that a polymer load of at least 3 wt %, and preferably at least 3.5 wt %, in the aqueous fluid leads to a template particle with a hydrogel having an effective mesh size less than 200 nm. In certain aspects, the polymer is an acrylamide/bisacrylamide copolymer matrix and the aqueous phase fluid includes at least 3.5 wt % of the matrix.

In an exemplary method, template particles with micropores can be manufactured by preparing an aqueous phase solution that includes hydrogel monomers and a porogen, such as polyethylene glycol (PEG). Preferably, the aqueous solution includes 4-20 wt % of PEG. As PEG and many hydrogel polymers, such as polyacrylamide, are not miscible, the hydrogel monomers should be added to the aqueous fluid as monomers rather than as polymers. The aqueous fluid, including the PEG and hydrogel monomers, are co-flowed with a fluid immiscible to the aqueous phase, e.g., an oil, through a droplet generation device. In certain aspects, phase separation begins concurrently with the polymerization of the hydrogel monomers. After polymerization, the PEG is washed away, and results in hydrogel template particles with micropores.

In certain aspects, temperature-responsive polymers, such as N-isoproplyacrylamide, are used and the droplets are collected above the lower critical solution temperature (LCST) to create polymer-rich and polymer-deficient domains within a droplet.

The Inventors discovered that the degree of microporosity in the hydrogel of a template particle can be controlled by modulating one or more factors, including the chemical structures and interactions of monomers and crosslinkers in the hydrogel, the molecular weight of the monomers and/or porogen, the ratio of polymer to porogen, the mechanism of pore generation (i.e., phase separation between polymer components, polymerization above the LCST), presence of gas formation agents, use of mini-emulsions, doublet emulsion within gel precursor droplets, and freeze-thaw treatment during gelation.

In certain aspects, the template particles comprise a hydrogel. In certain embodiments, the hydrogel is selected from naturally derived materials, synthetically derived materials and combinations thereof. Examples of hydrogels include, but are not limited to, collagen, hyaluronan, chitosan, fibrin, gelatin, alginate, agarose, chondroitin sulfate, polyacrylamide, polyethylene glycol (PEG), polyvinyl alcohol (PVA), acrylamide/bisacrylamide copolymer matrix, polyacrylamide/poly(acrylic acid) (PAA), hydroxyethyl methacrylate (HEMA), poly N-isopropylacrylamide (NIPAM), and polyanhydrides, poly(propylene fumarate) (PPF).

In certain aspects, the template particles and/or hydrogels that compose the particles are allowed to solidify by triggering a gelation mechanism, including, but not limited to, the polymerization or crosslinking of a gel matrix. For instance, polyacrylamide gels are formed by copolymerization of acrylamide and bis-acrylamide. The reaction is a vinyl addition polymerization initiated by a free radical-generating system. In certain aspects, agarose hydrogels undergo gelation by cooling the hydrogels below the gelation temperature.

The composition and nature of the template particles may vary. For instance, in certain aspects, the template particles may be microgel particles that are micron-scale spheres of gel matrix. In some embodiments, the microgels are composed of a hydrophilic polymer that is soluble in water, including alginate or agarose. In other embodiments, the microgels are composed of a lipophilic microgel.

In some embodiments, the template particles have an average volume, and a method as described herein includes shrinking the template particles to decrease the average volume. The shrinking may occur upon the application of an external stimulus, e.g., heat. For instance, the template particles may be encapsulated in a fluid by shearing, followed by the application of heat, causing the template particles to shrink in size. The monodisperse single-emulsion droplet or double-emulsion droplet or GUV will not shrink because the droplet volume is constant and dictated by the original size of the template particle, but the template particle within the droplet will shrink away from the surface of the droplet.

The template particles may be loaded with at least one reagent and/or sample, which may include one or more of cells, genes, drug molecules, therapeutic agents, particles, bioactive agents, osteogenic agents, osteoconductive agents, osteoinductive agents, anti-inflammatory agents, growth factors, fibroin derived polypeptide particles, nucleic acid synthesis reagents, nucleic acid amplification reagents, reverse transcription reagents, nucleic acid detection reagents, target particles, DNA molecules, RNA molecules, genomic DNA molecules, and combinations of the same. The template particles may be loaded with reagents that can be triggered to release a desired compound, e.g., a substrate for an enzymatic reaction. For instance, a double emulsion droplet can be encapsulated in the template particles that are triggered to rupture upon the application of a stimulus, e.g., heat. The stimulus initiates a reaction after the template particles have been encapsulated in an immiscible carrier phase fluid.

Template particles may be generated under microfluidic control, e.g., using methods described in U.S. Patent Application Publication No. 2015/0232942, the disclosure of which is incorporated by reference herein. Microfluidic devices can form emulsions consisting of droplets that are extremely uniform in size. The template particles generation process may be accomplished by pumping two immiscible fluids, such as oil and water, into a junction. The junction shape, fluid properties (viscosity, interfacial tension, etc.), and flow rates influence the properties of the template particles generated but, for a relatively wide range of properties, template particles of controlled, uniform size can be generated using methods like T-junctions and flow focusing. To vary template particle size, the flow rates of the immiscible liquids may be varied since, for T-junction and flow focus methodologies over a certain range of properties, template particle size depends on total flow rate and the ratio of the two fluid flow rates. To generate a template particle with microfluidic methods, the two fluids are normally loaded into two inlet reservoirs (e.g., syringes, pressure tubes) and then pressurized as needed to generate the desired flow rates (e.g., using syringe pumps, pressure regulators, gravity, etc.). This pumps the fluids through the device at the desired flow rates, thus generating droplet of the desired size and rate.

In some embodiments, template particles may be generated using parallel droplet generation techniques, including, but not limited to, serial splitting and distribution plates. Parallel droplet generation techniques of interest further include those described by Abate and Weitz, Lab Chip 2011, Jun. 7; 11(11):1911-5; and Huang et al., RSC Advances 2017, 7, 14932-14938; the disclosure of each of which is incorporated by reference herein.

In some embodiments, the template particles may be removed from the fluid, dried, and stored in a stable form for a period of time. Examples of drying approaches include, but are not limited to, heating, drying under vacuum, freeze drying, and supercritical drying. In some embodiments, the dried template particles may be combined with a fluid, but still retain the shape and structure as independent, often spherical, gel particles. In some embodiments, the dried template particles are combined with an appropriate fluid, causing a portion of the fluid to be absorbed by the template particles. In some embodiments, the porosity of the template particles may vary, to allow at least one of a plurality of target particles to be absorbed into the template particles when combined with the appropriate fluid. Any convenient fluid that allows for the desired absorption to be performed in the template particles may be used.

In certain aspects, a surfactant may be used to stabilize the template particles. Accordingly, a template particle may involve a surfactant stabilized emulsion, e.g., a surfactant stabilized single emulsion or a surfactant stabilized double emulsion. Any convenient surfactant that allows for the desired reactions to be performed in the template particles may be used. In other aspects, a template particle is not stabilized by surfactants or particles.

In some embodiments of the template particles, a variation in diameter or largest dimension of the template particles such that at least 50% or more, e.g., 60% or more, 70% or more, 80% or more, 90% or more, 95% or more, or 99% or more of the template particles vary in diameter or largest dimension by less than a factor of 10, e.g., less than a factor of 5, less than a factor of 4, less than a factor of 3, less than a factor of 2, less than a factor of 1.5, less than a factor of 1.4, less than a factor of 1.3, less than a factor of 1.2, less than a factor of 1.1, less than a factor of 1.05, or less than a factor of 1.01.

Monodisperse droplets may be effectively obtained by using capture particles to template the formation of droplets, which can include, e.g., monodisperse single-emulsion droplets, multiple-emulsion droplets, or Giant Unilamellar Vesicles (GUV)

As used herein, the term “monodisperse,” as applied to droplets, e.g., monodisperse single-emulsion droplets, refers to a variation in diameter or largest dimension of droplets produced by shearing in the presence of capture template particles, which is less than would occur when droplets are produced by shearing under the same conditions in the absence of the capture template particles. Generally, monodisperse single-emulsion droplets or multiple-emulsion droplets can have more variation in diameter or largest dimension as compared to the capture template particles from which they are generated, while still functioning in the various methods described herein. Monodisperse droplets generally range from about 0.1 to about 1000 μm in diameter or largest dimension, and may have a variation in diameter or largest dimension of less than a factor of 10, e.g., less than a factor of 5, less than a factor of 4, less than a factor of 3, less than a factor of 2, less than a factor of 1.5, less than a factor of 1.4, less than a factor of 1.3, less than a factor of 1.2, less than a factor of 1.1, less than a factor of 1.05, or less than a factor of 1.01, in diameter or the largest dimension. In some embodiments, monodisperse droplets have a variation in diameter or largest dimension such that at least 50% or more, e.g., 60% or more, 70% or more, 80% or more, 90% or more, 95% or more, or 99% or more of the monodisperse droplets, vary in diameter or largest dimension by less than a factor of 10, e.g., less than a factor of 5, less than a factor of 4, less than a factor of 3, less than a factor of 2, less than a factor of 1.5, less than a factor of 1.4, less than a factor of 1.3, less than a factor of 1.2, less than a factor of 1.1, less than a factor of 1.05, or less than a factor of 1.01. In some embodiments, monodisperse droplets have a diameter of about 1.0 μm to 1000 μm, inclusive, such as about 1.0 μm to about 750 μm, about 1.0 μm to about 500 μm, about 1.0 μm to about 250 μm, about 1.0 μm to about 200 μm, about 1.0 μm to about 150 μm, about 1.0 μm to about 100 μm, about 1.0 μm to about 10 μm, or about 1.0 μm to about 5 μm, inclusive.

In practicing the methods as described herein, the composition and nature of the monodisperse droplets, e.g., single-emulsion and multiple-emulsion droplets, may vary. For instance, in certain aspects, a surfactant may be used to stabilize the droplets.

Accordingly, a droplet may involve a surfactant stabilized emulsion, e.g., a surfactant stabilized single emulsion or a surfactant stabilized double emulsion. Any convenient surfactant that allows for the desired reactions to be performed in the droplets may be used. In other aspects, monodisperse droplets are not stabilized by surfactants.

The droplets described herein may be prepared as emulsions, e.g., as an aqueous phase fluid dispersed in an immiscible phase carrier fluid (e.g., a fluorocarbon oil, silicone oil, or a hydrocarbon oil) or vice versa. In particular, multiple-emulsion droplets as described herein may be provided as double-emulsions, e.g., as an aqueous phase fluid in an immiscible phase fluid, dispersed in an aqueous phase carrier fluid; quadruple emulsions, e.g., an aqueous phase fluid in an immiscible phase fluid, in an aqueous phase fluid, in an immiscible phase fluid, dispersed in an aqueous phase carrier fluid; and so on. Generating a monodisperse single-emulsion droplet or a multiple-emulsion droplet as described herein may be performed without microfluidic control. In alternative embodiments, a monodisperse single-emulsion may be prepared without the use of a microfluidic device, but then modified using a microfluidic device to provide a multiple emulsion, e.g., a double emulsion.

Monodisperse single emulsions may be generated without the use of microfluidic devices using the methods described herein. Producing a monodisperse emulsion using capture template particles can provide emulsions including droplets that are extremely uniform in size. The droplet generation process may be accomplished by combining a plurality of capture template particles with a first fluid to provide a first mixture, wherein the first fluid includes a plurality of target particles; combining the first mixture with a second fluid to provide a second mixture, wherein the second fluid is immiscible with the first fluid; and shearing the second mixture such that a plurality of the capture template particles are encapsulated in a plurality of monodisperse droplets in the second fluid, thereby providing a plurality of monodisperse droplets including the first fluid, one of the capture template particles, and one of the plurality of target particles. To vary droplet size, the shearing rate and capture template particle sizes may be varied. For agarose gels, the capture template particles can be liquefied using an external stimulus (e.g., heat) to generate a liquid monodisperse emulsion.

The percentage of monodisperse droplets, e.g., monodisperse single-emulsion droplets or multiple-emulsion droplets, with one, and not more than one, capture template particle may be about 70% or more; about 75% or more; about 80% or more; about 85% or more; about 90% or more; or about 95% or more. For example, the percentage of monodisperse droplets with one, and not more than one, capture template particle may be from about 70% to about 100%, e.g., from about 75% to about 100%, from about 80% to about 100%, from about 85% to about 100%, from about 90% to about 100%, or from about 95% to about 100%. As a further example, the percentage of monodisperse droplets with one, and not more than one, capture template particle may be from about 70% to about 95%, e.g., from about 75% to about 90%, or from about 80% to about 85%. The percentage of capture template particles that are encapsulated in monodisperse droplets in the second fluid may be about 70% or more; about 75% or; about 80% or more; about 85% or more; or about 90% or more. For example, the percentage of capture template particles that are encapsulated in monodisperse droplets in the second fluid may be from about 70% to about 100%, e.g., from about 75% to about 100%, from about 80% to about 100%, from about 85% to about 100%, from about 90% to about 100%, or from about 95% to about 100%. As a further example, the percentage of capture template particles that are encapsulated in monodisperse droplets in the second fluid may be from about 70% to about 95%, e.g., from about 75% to about 90%, or from about 80% to about 85%.

Double emulsions may also be generated without the use of microfluidic devices using the methods described herein. A double emulsion includes droplets contained within droplets, e.g., an aqueous phase fluid surrounded by an immiscible phase shell in an aqueous phase carrier fluid (e.g., water-in oil-in water) or a immiscible phase fluid surrounded by an aqueous phase shell in an immiscible phase carrier fluid (e.g., oil-in water-in oil). The second mixture described herein, which includes monodisperse single-emulsion droplets in the second fluid, is combined with a third fluid to produce a third mixture, wherein the third fluid is immiscible with at least the second fluid. The third mixture is then sheared to encapsulate the capture template particles in double-emulsion droplets in the third fluid. The third fluid may be immiscible with both the first and second fluids.

A particularly useful kind of double emulsion includes an aqueous droplet encapsulated within a slightly larger oil droplet, itself dispersed in a carrier aqueous phase. Double emulsions are valuable because the inner “core” of the structure can be used to provide active compounds, like dissolved solutes or biological materials, where they are shielded from the external environment by the surrounding oil shell. A benefit of generating double emulsions using capture template particles is similar to that for the generation of single emulsions, in that the double emulsion dimensions (inner and outer droplet sizes) can be controlled over a wide range and the droplets can be formed with a high degree of uniformity. As discussed herein, in suitable embodiments the capture template particles can be dissolved and/or melted within the monodisperse droplets. Accordingly, in some embodiments multiple emulsions, e.g., double emulsions, may be prepared from monodisperse droplets which no longer contain an intact template particle yet retain their original size. In this manner, such monodisperse droplets may serve as templates for the preparation of multiple emulsions, e.g., double emulsions.

Encapsulation in droplets of sample materials and/or reagents, e.g., nucleic acids and/or nucleic acid synthesis reagents (e.g., isothermal nucleic acid amplification reagents and/or nucleic acid amplification reagents), can be achieved via a number of methods, including microfluidic and non-microfluidic methods. In the context of microfluidic methods, there are a number of techniques that can be applied, including glass microcapillary double emulsification or double emulsification using sequential droplet generation in wettability patterned devices.

Microcapillary techniques form droplets by generating coaxial jets of the immiscible phases that are induced to break into droplets via coaxial flow focusing through a nozzle. However, a potential disadvantage of this approach is that the devices are generally fabricated from microcapillary tubes that are aligned and glued together. Since the drop formation nozzle is on the scale of tens of microns, even small inaccuracies in the alignment of the capillaries can lead to a device failure. By contrast, sequential drop formation in spatially patterned droplet generation junctions can be achieved in devices fabricated lithographically, making them simpler to build and to create in large numbers while maintaining uniformity over dimensions.

However, in some cases the planar nature of these devices may not be ideal for generating double emulsions, since the separate phases all enter the device while in contact with the channel walls, necessitating that wettability be carefully patterned to enable engulfment of the appropriate phases at the appropriate locations. This may make the devices more difficult to fabricate, and in some cases, may prevent emulsification of liquids whose wetting properties are not optimized for the device. Accordingly, in some aspects the present disclosure provides methods for generating a monodisperse emulsion which encapsulates sample materials and/or reagents, e.g., nucleic acids and/or nucleic acid synthesis reagents (e.g., isothermal nucleic acid amplification reagents and/or nucleic acid amplification reagents) without the use of a microfluidic device.

For example, the methods as described herein may include combining a plurality of capture template particles with a first fluid to provide a first mixture, wherein the first fluid includes a plurality of target particles, e.g., nucleic acids, etc. In some embodiments, the combining the plurality of capture template particles with the first fluid to provide the first mixture includes causing a portion of the first fluid, and the target particles and/or reagents contained therein, to be absorbed, or attached, by the capture template particles. In some embodiments, combining the plurality of capture template particles with the first fluid to provide the first mixture includes flowing a portion of the first fluid into the capture template particles. In some embodiments, combining the plurality of capture template particles with the first fluid to provide the first mixture includes diffusing a portion of the first fluid into the capture template particles. In some embodiments, the combining the plurality of capture template particles with the first fluid to provide the first mixture includes swelling the capture template particles with a portion of the first fluid.

As discussed herein, the disclosed template particles can be used in methods that generally involve combining a plurality of capture template particles with a first fluid to provide a first mixture, wherein the first fluid includes a plurality of target particles. This first mixture is combined with a second fluid immiscible with the first fluid to provide a third mixture. The third mixture is sheared such that a plurality of the capture template particles are encapsulated in a plurality of monodisperse droplets in the second fluid, thereby providing a plurality of monodisperse droplets including the first fluid, one of the capture template particles, and one of the plurality of target particles. In some embodiments, the methods include the further step of combining a third fluid with the third mixture, following the shearing of the third mixture, to produce a fourth mixture, wherein the third fluid is immiscible with the second fluid.

The first fluid is generally selected to be immiscible with the second fluid and share a common hydrophilicity/hydrophobicity with the material which constitutes the capture template particles. The third fluid is generally selected to be immiscible with the second fluid, and may be miscible or immiscible with the first fluid.

Accordingly, in some embodiments, the first fluid is an aqueous phase fluid; the second fluid is a fluid which is immiscible with the first fluid, such as a non-aqueous phase, e.g., a fluorocarbon, silicone oil, oil, or a hydrocarbon oil, or a combination thereof; and the third fluid is an aqueous phase fluid. Alternatively, in some embodiments the first fluid is a non-aqueous phase, e.g., a fluorocarbon oil, silicone oil, or a hydrocarbon oil, or a combination thereof; the second fluid is a fluid which is immiscible with the first fluid, e.g., an aqueous phase fluid; and the third fluid is a fluorocarbon oil, silicone oil, or a hydrocarbon oil or a combination thereof.

The non-aqueous phase may serve as a carrier fluid forming a continuous phase that is immiscible with water, or the non-aqueous phase may be a dispersed phase. The non-aqueous phase may be referred to as an oil phase including at least one oil, but may include any liquid (or liquefiable) compound or mixture of liquid compounds that is immiscible with water. The oil may be synthetic or naturally occurring. The oil may or may not include carbon and/or silicon, and may or may not include hydrogen and/or fluorine. The oil may be lipophilic or lipophobic. In other words, the oil may be generally miscible or immiscible with organic solvents. Exemplary oils may include at least one silicone oil, mineral oil, fluorocarbon oil, vegetable oil, or a combination thereof, among others.

In exemplary embodiments, the oil is a fluorinated oil, such as a fluorocarbon oil, which may be a perfluorinated organic solvent. Examples of a suitable fluorocarbon oils include, but are not limited to, C9H5OF15 (HFE-7500), C21F48N2 (FC-40), and perfluoromethyldecalin (PFMD).

In certain aspects, the first fluid includes a plurality of target particles (e.g. DNA molecules such as genomic DNA molecules, RNA molecules, nucleic acid synthesis reagents such as nucleic acid amplification reagents including PCR and/or isothermal amplification reagents).

Gelling agents may be added to solidify the outer layers of the droplet. Gelling agents include, but are not limited to, gelatin, agar, xanthan gum, gellan gum, carrageenan, isubgol, and guar gum.

In certain aspects, a surfactant may be included in the first fluid, second fluid, and/or third fluid. Accordingly, a droplet may involve a surfactant stabilized emulsion, e.g., a surfactant stabilized single emulsion or a surfactant stabilized double emulsion, where the surfactant is soluble in the first fluid, second fluid, and/or third fluid. Any convenient surfactant that allows for the desired reactions to be performed in the droplets may be used, including, but not limited to, octylphenol ethoxylate (Triton X-100), polyethylene glycol (PEG), C26H50O10 (Tween 20) and/or octylphenoxypolyethoxyethanol (IGEPAL). In other aspects, a droplet is not stabilized by surfactants.

The surfactant used depends on a number of factors such as the oil and aqueous phases (or other suitable immiscible phases, e.g., any suitable hydrophobic and hydrophilic phases) used for the emulsions. For example, when using aqueous droplets in a fluorocarbon oil, the surfactant may have a hydrophilic block (PEG-PPO) and a hydrophobic fluorinated block (Krytox® FSH). If, however, the oil was switched to a hydrocarbon oil, for example, the surfactant may instead be chosen such that it had a hydrophobic hydrocarbon block, like the surfactant ABIL EM90.

Other surfactants can also be envisioned, including ionic surfactants. Other additives can also be included in the oil to stabilize the droplets, including polymers that increase droplet stability at temperatures above 35° C.

Exemplary surfactants which may be utilized to provide thermostable emulsions are the “biocompatible” surfactants that include PEG-PFPE (polyethyleneglycol-perflouropolyether) block copolymers, e.g., PEG-Krytox® (see, e.g., Holtze et al., “Biocompatible surfactants for water-in-fluorocarbon emulsions,” Lab Chip, 2008, 8, 1632-1639, the disclosure of which is incorporated by reference herein), and surfactants that include ionic Krytox® in the oil phase and Jeffamine® (polyetheramine) in the aqueous phase (see, e.g., DeJournette et al., “Creating Biocompatible Oil-Water Interfaces without Synthesis: Direct Interactions between Primary Amines and Carboxylated Perfluorocarbon Surfactants”, Anal. Chem. 2013, 85(21):10556-10564, the disclosure of which is incorporated by reference herein). Additional and/or alternative surfactants may be used provided they form stable interfaces. Many suitable surfactants will thus be block copolymer surfactants (like PEG-Krytox®) that have a high molecular weight. These examples include fluorinated molecules and solvents, but it is likely that non-fluorinated molecules can be utilized as well. The term “surfactant” refers to any molecule having both a polar head group, which energetically prefers solvation by water, and a hydrophobic tail that is not well solvated by water. The presently disclosed methods are not limited to a particular surfactant. A variety of surfactants are contemplated including, but not limited to, nonionic and ionic surfactants (e.g., TRITON X-100; TWEEN 20; and TYLOXAPOL) or combinations thereof.

To generate a monodisperse emulsion, the disclosed methods include a step of shearing the second mixture provided by combining the first mixture with a third fluid immiscible with the first fluid. Any suitable method or technique may be utilized to apply a sufficient shear force to the second mixture. For example, the second mixture may be sheared by flowing the second mixture through a pipette tip. Other methods include, but are not limited to, shaking the second mixture with a homogenizer (e.g., vortexer), or shaking the second mixture with a bead beater. The application of a sufficient shear force breaks the second mixture into monodisperse droplets that encapsulate one of a plurality of capture template particles. There may also be some droplets that do not contain one of the plurality of capture template particles.

Generally, if the shear is increased, the average droplet size generated will be lower than that of the size of the capture template particles. However, since the capture template particles are in solid form, the droplets containing them will not be any smaller in size, thereby generating a monodisperse emulsion. If the shear rate is substantially higher than the modulus of the capture template particle, then the shear can squeeze liquid out of the capture template particles. Without intending to be bound by any particular theory, it is proposed that a suitable shear rate is one which matches appropriately the modulus of the capture template particles. For example, it may be desirable to select a shear rate/force higher than the Laplace pressure of the droplets of the desired size but less than the modulus of the template particles.

### EXAMPLES

### Example 1

### Template Particles with Nanoporous Structure

Template particles were manufactured using an acqueous phase fluid that included 3.5 wt % of polyacrylamide (PAA) in accordance with the methods disclose herein. The Inventors discovered that polymer loading amounts less than about 3.0 wt % in the aqueous phase fluid failed to lead to gelation necessary to create the particles.

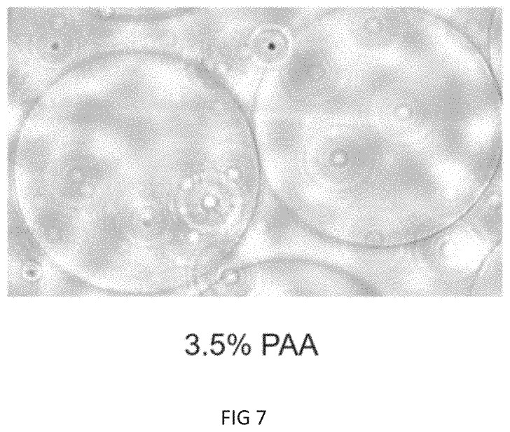

FIG. 7 provides a microscope image of the resulting 3.5% PAA template particles with a nanoporous structure in the hydrogel.

### Example 2

### Template Particles with a Microporous Structure

Template particles were manufactured in accordance with the methods provided herein using varying concentrations of acrylamide monomers and PEG (as a porogen). Additionally, template particles were manufactured using PEG of varying molecular weights. The Inventors discovered that using PEG with a higher molecular weight resulted in template particles with a more hollowed microporous structure and included pores of larger sizes.

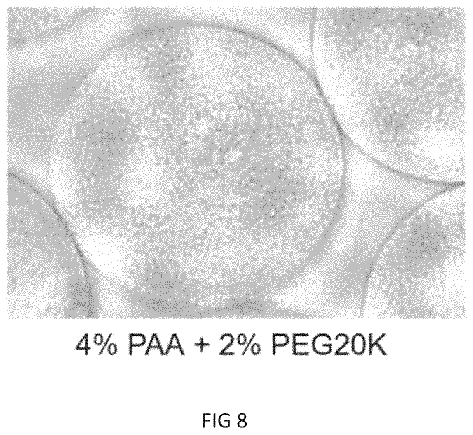

FIG. 8 provides a microscope image of a resulting template particle with a microporous structure made using 4% PAA and 2% PEG20K.

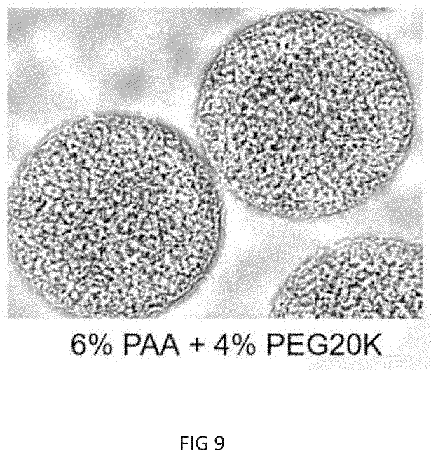

FIG. 9 provides a microscope image of a resulting template particle with a microporous structure made using 6% PAA and 4% PEG20K.

### Example 3

### Manufacturing Efficiency

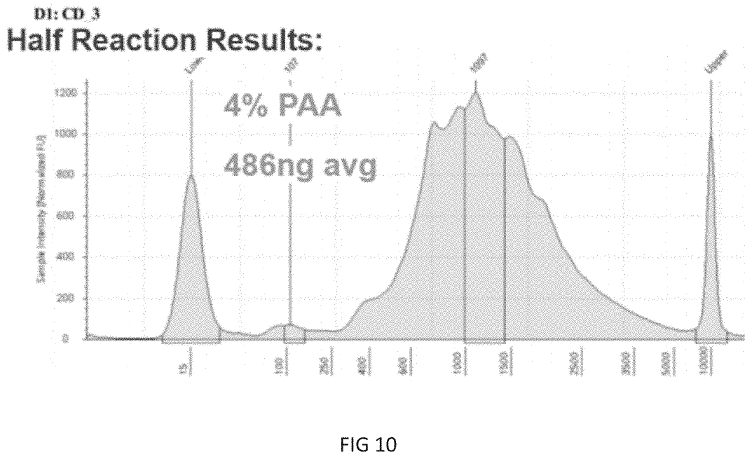

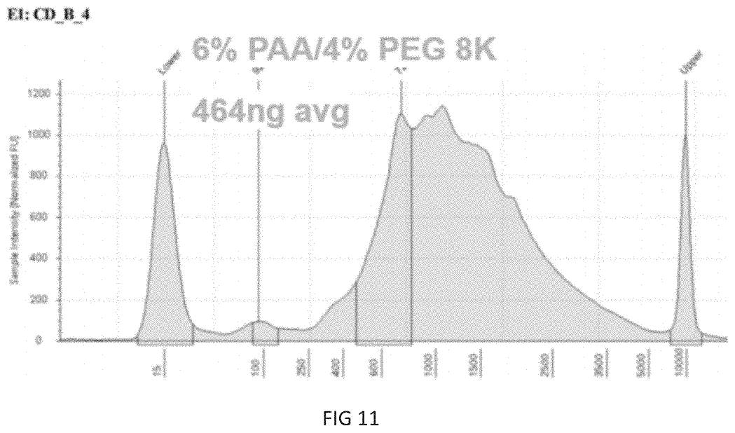

FIGS. 10-11 show the results of manufacturing template particles with either a nanoporous structure (4% PAA) or a microporous structure (6% PAA/4% PEG8K). The manufacture of each type of particle produced templates with a nominal size distribution that averaged approximately 1400 ng. This size and size distribution aligns with template particles manufactured without micro- or nanopores.

### Example 4

### Nucleic Acid Amplification and Sequencing

Template particles were manufactured that had a nanoporous structure (4% PAA) or a microporous structure (6% PAA/4% PEG8K) or no micro- or nanoporous structure (8% PAA). The template particles were emulsified into monodispersed droplets with cells and reagents required for whole transcriptome amplification. Nucleic acids from the cells were captured by the template particles, barcoded with UMIs, reverse transcribed and amplified. The amplified nucleic acids were recovered and sequenced.

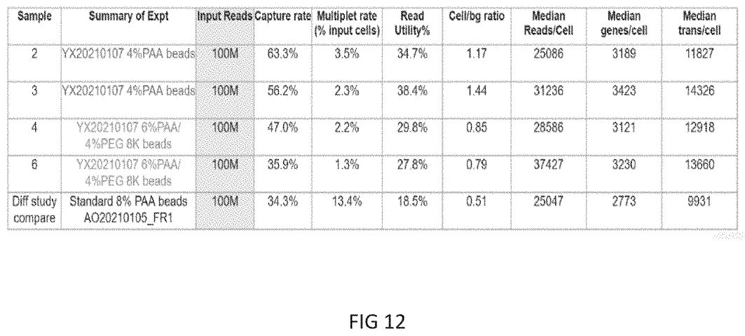

FIG. 12 shows the results of this assay across several critical steps. The table in FIG. 12 reveals that template particles with nano- or micropores successfully captured targets (i.e., cells) more often than pores without pores, and thus had a higher capture rate. The template particles with nano- and micropores were also able to provide an increased cell/background ratio median reads per cell, median genes read per cell, and median transcripts per cell.

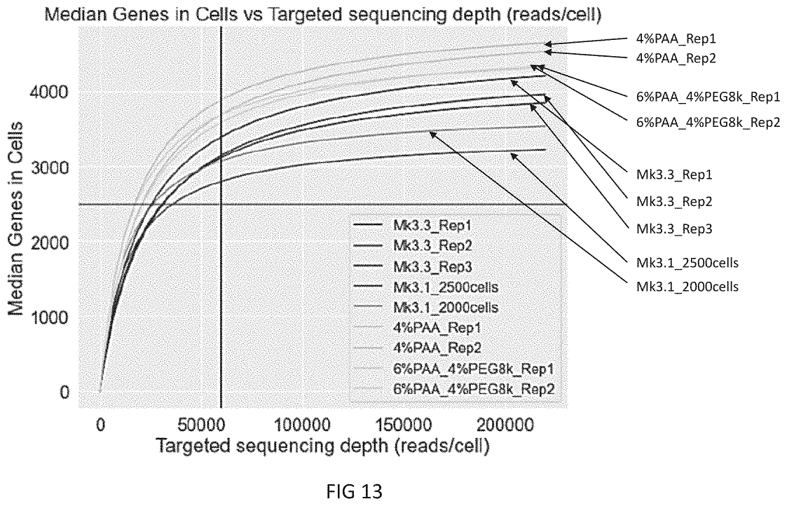

FIG. 13 provides a saturation curve comparing the median genes read per cell versus targeted sequencing depth (reads/cell).

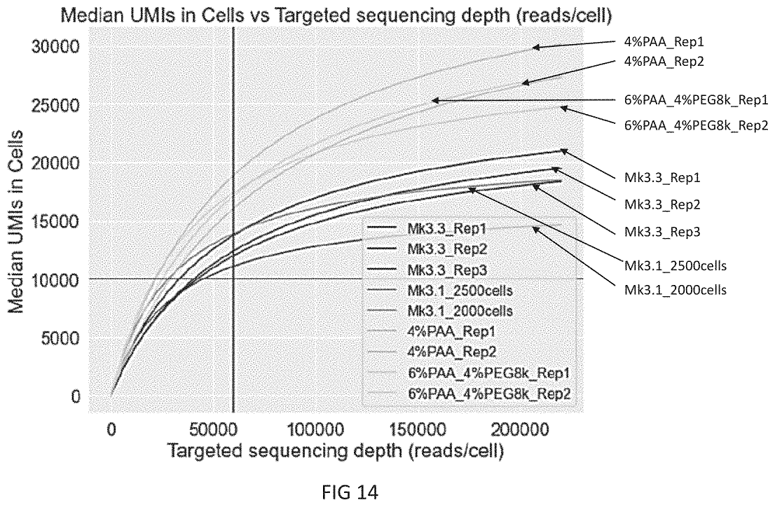

FIG. 14 provides a saturation curve comparing the median UMIs read per cell versus targeted sequencing depth (reads/cell).

The results in FIGS. 13-14 show that template particles with micro- or nanopores outperform those without pores (the “MK3” results) at a lower sequencing depth. Further, these results surpass those required for a minimum viable product.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A composition comprising:
a plurality of hydrogel particles suspended in an aqueous liquid, each hydrogel particle comprising a mesh of cross-linked polymers, wherein each hydrogel particle comprises:
(i) a plurality of micropores extending through the mesh of cross-linked polymers, each micropore having an open interior volume having a dimension of at least about a micron; and/or
(ii) a nanoporous structure in the mesh of cross-linked polymers, wherein the hydrogel mesh has a mesh size of at least about 200 nm, wherein the nanoporous structure comprises at least one open interior volume in the hydrogel mesh;
wherein the particle is loaded with a reaction reagent; and
wherein the aqueous liquid permeates the interior volume of the micropores and/or nanoporous structure allowing analytes in the fluid to access the reagent.

2. The composition of claim 1, wherein each particle comprises a plurality of micropores.

3. The composition of claim 2, wherein each particle further comprises a nanoporous structure.

4. The composition of claim 1, wherein each particle comprises a nanoporous structure.

5. The composition of claim 1, wherein the reagents comprise one or more of enzymes, enzyme cofactors, nucleotides, polynucleotides, amino acids, peptides, proteins, probes, primers, salts, ions, buffers, labels, dyes, antibodies, polymers, and carbohydrates.

6. The composition of claim 5, wherein the reagents comprise one or more target capture moiety.

7. The composition of claim 6, wherein the target capture moiety captures one or more of circulating cells, cellular components, cell-free nucleic acids, extracellular vesicles, protein antigens, prokaryotic cells, fungi, viruses, and combinations thereof.

8. The composition of claim 5, wherein the reagents comprise reagents for one or more of nucleic acid synthesis, transcription, reverse transcription, and cell lysis.

9. The composition of claim 5, wherein one or more of the reagents are covalently linked to the particle.

10. A method for performing a bioassay, the method comprising:
combining template particles with samples in a first fluid, wherein the template particles are hydrogel particles suspended in the first fluid, each hydrogel particle comprising a mesh of cross-linked polymers, wherein each hydrogel particle is loaded with a reaction reagent and comprises:
(i) a plurality of micropores extending through the mesh of cross-linked polymers, each micropore having an open interior volume having a dimension of at least about a micron; and/or
(ii) a nanoporous structure in the mesh of cross-linked polymers, wherein the hydrogel mesh has a mesh size of at least 200 nm in length, wherein the nanoporous structure comprises at least one open interior volume in the hydrogel mesh;
adding a second fluid immiscible to the first fluid; and
shearing the fluids to generate a plurality of monodispersed droplets simultaneously that contain a single one of the template particles and one or more of the samples.

11. The method of claim 10, wherein the first liquid permeates the interior volume of the micropores and/or nanoporous structure allowing analytes in the first fluid to access the reaction reagent.

12. The method of claim 11, wherein in the monodisperse droplets, the interior volume is occupied by at least the first fluid and one or more of the reagents.

13. The method of claim 10, wherein the samples comprise at least one of circulating cells, cellular components, cell-free nucleic acids, extracellular vesicles, protein antigens, prokaryotic cells, fungi, viruses, and combinations thereof.

14. The method of claim 13, wherein the samples are cells and the sample in each of the plurality of monodisperse droplets is a single cell.

15. A method for producing cross-linked template particles, the method comprising:
preparing an aqueous phase fluid comprising an acrylamide/bisacrylamide copolymer matrix, wherein the aqueous phase fluid comprises at least about 3.5 wt % acrylamide/bisacrylamide; and
co-flowing the aqueous phase fluid and a fluid immiscible to the aqueous phase fluid through a droplet generation device,
wherein the resulting template particles have an effective hydrogel mesh size less than 200 nm in length.

17. The method of claim 16, wherein the effective hydrogel mesh size is modulated via a ratio of acrylamide monomers to bisacrylamide monomers in the copolymer matrix.

18. A method for producing cross-linked template particles, the method comprising:
preparing an aqueous phase fluid comprising acrylamide monomers and PEG; and
co-flowing the aqueous phase fluid and a fluid immiscible to the aqueous phase fluid through a droplet generation device,
wherein the resulting template particles comprise micropores having an open interior volume having a dimension of at least about a micron.

19. The method of claim 18, wherein the aqueous phase fluid comprises at least about 4 wt % and 2 wt % PEG.

20. The method of claim 18, further comprising washing the PEG from the template particles.
